(19) United States
(12) Patent Application Publication
(10) Pub. No.: US 2023/0181886 A1
(43) Pub. Date: Jun. 15, 2023

(54) FLUID INJECTION DEVICE FOR INJECTING A FLUID BEHIND THE TYMPANIC MEMBRANE

(71) Applicant: STICHTING HET NEDERLANDS KANKER INSTITUUT-ANTONI VAN LEEUWENHOEK ZIEKENHUIS, AMSTERDAM (NL)

(72) Inventors: Charlotte Louise ZUUR, AMSTERDAM (NL); Boudewijn VAN LIMPT, SCHIPHOL-RIJK (NL)

(21) Appl. No.: 17/923,671

(22) PCT Filed: May 7, 2021
(86) PCT No.: PCT/NL2021/050297

§ 371 Date: Nov. 7, 2022

(30) Foreign Application Priority Data
May 8, 2020 (NL) 2025530

(51) Int. Cl. A61M 31/00; A61F 11/00

(52) U.S. Cl. CPC A61M 31/00; A61F 11/00; A61M 2205/502; A61M 2210/0668; A61M 2025/0091

(57) ABSTRACT

Fluid injection device for injecting a fluid behind a tympanic membrane of a patient, comprising: a hollow needle having a needle tip with a fluid outlet at a distal end thereof, the needle tip being configured for piercing the tympanic membrane of the patient; a container for storing the fluid to be injected behind the tympanic membrane, the container being arranged in fluid communication with the hollow needle; characterised, in that the hollow needle comprises an elongated venting aperture in an outer wall thereof, the elongated venting aperture being positionable at an inner side of an ear membrane of the patient as well as at an outer ide of said ear membrane when injecting fluid, so as to allow excess fluid on the inner side of said membrane to flow through the elongated venting aperture, to the outer side of said membrane.

The present invention relates to a fluid injection device for injecting a fluid behind a tympanic membrane of a patient, a hollow needle for use in combination with such a fluid injection device, as well as an injection assembly comprising such a fluid injection device and an injection fluid.

Behind the eardrum we find the cavum tympani, the middle ear, which is a semi-closed cavity filled with air and an inner lining of mucosa. It contains the auditory ossicles that transfer sound from the eardrum up to the oval window, where sound is transmitted via the ductus cochlearis to the auditory nerve and brains. A small increase of pressure in the middle ear will cause tension on the tympanic membrane and pain symptoms to patients. A larger increase in pressure of the middle ear may cause damage to the oval or round window and/or of the ductus cochlearis, a so-called barotrauma, leading to potentially permanent (irreversible) hearing loss or deafness of the patient.

Transtympanic or intratympanic injection means injecting a fluid through or via the ear drum into the middle ear and typically takes place while the patient is fully awake. During this procedure the pressure in the middle ear, behind the tympanic membrane, increases. As a result of the increasing pressure in the middle ear this operation is often quite painful for patients and may cause a barotrauma. One way to prevent this is to make two insertions (2 punctures, 2 holes) in the tympanic membrane: one to inject fluid and one to vent the middle ear. However, making two punctures in the tympanic membrane leads to an increased risk of inducing a permanent hole in the eardrum. Also, the fluid that is injected via the first hole, may immediately flow back via the second hole in the ear drum, leaving a therapeutically inadequate amount of liquid behind in the middle ear.

US 2005/0182385 A1 discloses a system for the sustained delivery of a medical liquid through the tympanic membrane and into the middle ear. In one embodiment described in US 2005/0182385 A1, a device includes a needle having a tip and a fluid outlet. The needle comprises two lumens, which are arranged coaxially. Via the inner lumen, a central bore and the fluid outlet, fluids can be delivered to the middle ear. Via inlets arranged in the outer wall of the needle, fluids can be withdrawn from the middle ear. The inlets, in use, are placed inside the middle ear, behind the tympanic membrane.

A need therefore remains for a device which is minimally invasive for the patient, and which has optimal venting to prevent pain symptoms and/or the risk for barotrauma. In addition, the device is preferably as simple as possible, to avoid room for mistakes, and induces as few vibrations as possible to prevent an undesired damage to the external ear (ear canal, ear drum) and middle ear (ossicles, oval window).

It is therefore an object of the present invention to provide a fluid injection device which improves at least one of the above-mentioned characteristics of fluid injection devices.

Accordingly, a first aspect of the invention relates to a fluid injection device for injecting a fluid behind a tympanic membrane of a patient, comprising:
a hollow needle having a needle tip with a fluid outlet at a distal end thereof, the needle tip being configured for piercing the tympanic membrane of the patient;
a container for storing the fluid to be injected behind the tympanic membrane, the container being arranged in fluid communication with the hollow needle;

characterised, in that the hollow needle comprises an elongated venting aperture in an outer wall thereof, the elongated venting aperture being positionable at an inner side of an ear membrane of the patient as well as at an outer side of said ear membrane when injecting fluid, so as to allow excess fluid on the inner side of said membrane to flow through the elongated venting aperture, to the outer side of said membrane.

The fluid injection device is for injection of a fluid in the middle ear or inner ear of a patient. As is common in patent literature, the word “fluid” refers here to either a gas or a liquid. The insertion fluid will typically be a liquid comprising a pharmaceutical agent. For example the fluid may be injected via the ear drum into the middle ear of a patient and/or via the oval window or round window in the inner ear of the patient. In another example, the fluid may be injected via tympanoplasty, a surgical technique under local or general anesthesia. In a yet further example, the injection in the middle ear can be effected through the Eustachian tube, the membrane between the Eustachian tube and the middle ear for that purpose also being defined as an ear membrane. In embodiments, the fluid is injected in the middle ear of the patient, the needle only penetrating or piercing the tympanic membrane. In other embodiments, the fluid is injected in the inner ear of the patient, the needle penetrating or piercing both the tympanic membrane and the oval window and/or the round window. Hence, the wording “behind the tympanic membrane”, as used herein, refers e.g. to into the middle ear and/or into the inner ear.

In embodiments of the fluid injection device according to the invention, the needle is minimally invasive; only one relatively small insertion hole needs to be made in the tympanic membrane to result in a successful injection of fluid without pain symptoms due to increased tension on the eardrum and without the risk for barotrauma due to increased pressure of the middle ear. This is particularly the case when the needle is single-walled and comprises only an injection channel.

In embodiments of the fluid injection device according to the invention, optimal venting of the middle ear and/or inner ear is achieved, as the elongated venting channel extends along both sides of the eardrum (membrane) This allows a very easy flow of fluid from inside of the membrane to outside of the membrane, without the flow being hindered by the physical dimensions of an outflow channel. It is noted that the wording ‘inside’ and ‘outside’ is relative to the eardrum of the patient. Therefore, an excessive pressure increase inside the middle ear leading to pain symptoms and/or barotrauma of the inner ear is prevented. Likewise, an excessive pressure increase inside the inner ear may be prevented. This optimal venting may especially be advantageous when the fluid is relatively viscous. Using a relative viscous fluid as injection fluid behind the tympanic membrane is preferred as this allows the fluid to fold to the structure of the middle ear, which is expected to result in optimal contact surface and in relatively fast absorption of the liquid.

Advantageously, the fluid injection device, or at least the part thereof that is inserted in the ear, is simple and has relatively few components. This reduces the room for making mistakes causing damage to the ear and hearing dysfunction.

It is found by the applicant that injecting a fluid behind an ear membrane is beneficial for all kinds of hearing defects and other therapeutic indications. For example, injecting a fluid by using the fluid injection device according to the invention is beneficial in the prevention, alleviation, or curing of hearing loss. For example the applicant found that injecting the fluid containing a pharmaceutical agent may be beneficial in the prevention, alleviation, or curing of hearing loss. Preferably, the injection fluid may prevent, alleviate or cure sensorineural hearing loss. Sensorineural hearing loss is a disease which may e.g. be caused by an infection, by age, by genetic mutations, by noise, etc. Sensorineural hearing loss is also observed as a side-effect when treating patients for cancer using pharmaceutical agent comprising platinum-based chemotherapy, or more in particular cisplatin or carboplatin-based chemo therapy. More specifically, it is observed by the present applicant that injecting sodium thiosulphate behind the tympanic membrane of the patient, i.e. intratympanically, can help to prevent platinum-induced, such as cisplatin-induced or carboplatin-induced sensorineural hearing loss.

The hollow needle of the fluid injection device is configured for piercing at least the tympanic membrane of the patient. The needle is inserted in the outer ear canal of the patient, e.g. by a surgeon or physician assistant, and penetrates or pierces the tympanic membrane such that the needle tip reaches the middle ear. When the fluid is to be injected in the middle ear of the patient, the needle tip typically only penetrates the tympanic membrane. When the fluid is to be injected in the inner ear of the patient, the needle tip is inserted further in the ear of the patient, and also penetrates the round window and/or the oval window.

In embodiments the needle is used only once, and for each treatment a new needle is used. When both ears of a patient are to be treated, the needle may be used only once, such that two needles are needed for the treatment of both ears.

The amount of fluid stored in the container may e.g. be sufficient for a single treatment, e.g. an injection in one ear of one patient. Alternatively, the amount of fluid stored in the container may e.g. be sufficient for two treatments, e.g. an injection in two ears of one patient. Alternatively, the amount of fluid stored in the container may be sufficient for several treatments, of several patients. The fluid communication between the hollow needle and the container may e.g. be embodied by a tubing. The amount of fluid stored in the container may be dependent on the length of the tubing, to account for any fluid remaining in the tubing and not injected in the ear.

The hollow needle comprises an elongated venting aperture in an outer wall thereof. When the fluid injection device is used by a user, e.g. a surgeon such as an ENT-surgeon (ear nose throat surgeon), i.e. otorhinolaryngologist, or a physician assistant, the elongated venting hole is positioned at both sides of the ear membrane.

In one example, when the fluid is to be injected in the middle ear, the elongated venting aperture is positioned at both sides of the tympanic membrane. That is, the elongated venting aperture is positioned at an inner side of the tympanic membrane, in the middle ear, and at an outer side of the tympanic membrane, in the outer ear canal. The middle ear is usually filled with air, i.e. a fluid. When an injection fluid, e.g. a liquid, e.g. comprising a pharmaceutical agent, is injected into the middle ear, the pressure inside the middle ear rapidly rises. Any excess fluid, e.g. air and/or injection fluid, in the middle ear can escape the middle ear by flowing out of the middle ear through the elongated venting aperture. This way, a rapid increase in pressure in the middle ear is prevented, preventing pain symptoms and the risk for barotrauma. Furthermore, the procedure is less painful for the patient.

Even though the middle ear is a semi-closed cavity and the Eustachian tube to a certain extent regulates the pressure in the middle ear, it cannot cope with a sudden pressure increase effected by injecting fluid therein. The elongated venting aperture(s) in the needle significantly improve this pressure regulating function by allowing fluid to move out of the middle ear. Also, the Eustachian tube may be blocked for all kinds of reasons, which prevents it from regulating the pressure in the middle ear.

In another example, when the fluid is to be injected in the inner ear, the elongated venting aperture is positioned at both sides of the round window and/or at both sides of the oval window. That is, the elongated venting aperture is positioned at an inner side of the round window and/or the oval window, in the inner ear, and at an outer side of the round window and/or the oval window, in the middle ear. The inner ear is usually filled with a liquid, i.e. a fluid. When an injection fluid, e.g. a liquid, e.g. comprising a pharmaceutical agent, is injected into the inner ear, the pressure inside the inner ear rapidly rises. Any excess fluid, e.g. air and/or injection fluid, in the inner ear can escape the inner ear by flowing out of the inner ear through the elongated venting aperture and towards the middle ear. This way, inner ear fluid can escape the inner ear leaving room for an adequate amount of therapeutic liquid to be placed in the inner ear.

The fluid injection device may further comprise a pump for pumping the fluid from the container to the needle, e.g. through tubing. Alternatively, the fluid injection device may e.g. comprise a plunger that can be activated by a hand and/or a finger of a person to transport the fluid from the container to the needle, e.g. through tubing. Other ways to transfer fluid from the container to the needle by hand are however also known. Alternatively, the needle and the container may be formed as a single part, such that the container and the needle tip are in direct connection with each other.

The fluid injection device may further comprise a grip, configured to allow a user of the fluid injection device to hold the needle steadily and stably. When the needle can be held stably and steadily, less vibrations are induced in the needle and optimal working conditions are provided to the user of the fluid injection device.

In an embodiment, the hollow needle comprises at least two elongated venting apertures, wherein a distance between a first elongated venting aperture and the fluid outlet differs from a distance between a second elongated venting aperture and the fluid outlet. The at least two elongated venting apertures, in one exemplary embodiment, may both or all be arranged at or near the same ear membrane, e.g. the tympanic membrane, of the patient when the fluid injection device is used. Depending on the insertion depth of the needle in the ear, more or less of the elongated venting apertures will perform a venting function. This allows e.g. the ENT-surgeon to control the amount of venting while injecting injection fluid. For example depending on the type of injection fluid used, more or less venting may be required. More specifically, the ENT-surgeon or other person using the injection device may choose to position one, two, or more elongated venting apertures (partially) through the respective ear membrane.

In another exemplary embodiment, the hollow needle may e.g. comprise one (at least one) elongated venting hole for venting a window separating the inner ear from the middle ear (the round window and/or the oval window) as well as one (at least one) elongated venting aperture for venting the eardrum separating the middle ear from the outer ear canal (i.e. the tympanic membrane). This allows inner ear fluid to escape the inner ear leaving room for an adequate amount of therapeutic liquid to be placed in the inner ear.

In an embodiment, the venting aperture is arranged in the longitudinal direction of the needle. This provides an operator of the fluid injection device with some margin regarding the exact positioning of the elongated venting aperture. Alternatively, the elongated venting aperture may be arranged at an angle with respect to the longitudinal direction of the needle. Preferably, the absolute value of said angle is between 0 and 60 degrees.

In an embodiment, the hollow needle is a single-walled needle. Advantageously, such a needle is relatively easily manufactured, as well as minimally invasive for the ear membrane to be pierced.

In an embodiment, the hollow needle is a double-walled needle having an inner wall and an outer wall, a fluid injection channel being defined by the inner wall and a venting channel being defined between the outer wall and the inner wall. Preferably, at the distal end of the needle near the fluid outlet, the needle is single-walled and has a relatively small diameter, while the double-walled structure is introduced by gradually increasing the diameter of the needle and providing a venting channel around the inner wall of the needle. This invades the ear membrane the least.

In an embodiment, the hollow needle is releasably coupled with a grip. This allows e.g. to re-use the grip and other parts of the device when using it for a second patient (or a second ear of the same patient), while the needle may be replaced. As the needle penetrates inside the body of a patient, regulations often stipulate that it may be used only once, or it should alternatively be cleaned extensively, including sterilization, before re-using it.

In an embodiment the needle, at or near an area where a or the aperture is arranged, comprises a positioning indicator for indicating a pre-defined insertion depth of the needle. For example, the positioning indicator may radially extend with respect to the needle, and may e.g. be a band, such as a coloured band or a rim. Alternatively, the positioning indicator may e.g. be a coloured mark on the needle. For example, seen from a distal end of the needle, the part of the needle that is to be inserted through the ear membrane may have a metal colour, while the part of the needle that is to remain outside of the ear membrane may be coated with a colour. This visually distinguishes these two parts from each other and provides instructions to the user of the fluid injection device regarding the optimal insertion depth. The coloured portion in principle may have any width and is preferably well visible for the person using the fluid injection device. For example, it may be a line spanning the entire circumference of the needle. In another example, the portion of the needle that is to penetrate the ear membrane of the patient may have a green colour, while the portion of the needle that is to remain in the ear canal of the patient may have a red colour. Optionally, in between the green and the red portion an orange portion may be present. Additionally, these distinguishing colour zones may be based upon anatomic standard measurements for children or adults in order to improve administration accuracy.

In an embodiment, the fluid injection device further comprises a pump for pumping the fluid from the container into the hollow needle and the grip comprises a touch button for activating the pump. Advantageously, a touch button on the grip in communication with a pump allows the person using the injection device to be in control of the dispensing of the fluid. Compared to any other button, the use of a touch button is advantageous as the use of a touch button induces the least amount of vibrations in the fluid injection device/needle, while still being practical in use. Any mechanically operated button, such as a click or slide button, will inherently introduce a vibration in the needle, and may thus be harmful for the ear.

In contrast to known methods carried out by a single person, wherein often use is made of a syringe for the injection of fluid, a (touch-)button activated pump decouples the need for the user to simultaneously steady the needle in both depth and lateral movement aspects, while applying substantial pressure to the syringe plunger. This improves delivery accuracy and reduces undesirable movements which can undermine the results of the procedure. This improved aspect is especially useful for injection of fluids which have a relatively high viscosity, as such fluids require higher injection forces.

Further advantageously, compared to the current methods wherein a second person, e.g. an assistant, is required to dispense fluid, the use of a touch button reduces the number of persons needed for the procedure and, maybe even more importantly, gives the person handling the fluid injection device direct control over the dispensing of the fluid, without any possible communication delays and/or errors.

The use of a touch button for activating the pump is also advantageous in fluid injection devices that do not have elongated venting apertures in accordance with the first aspect of the invention.

Accordingly, a second aspect of the invention relates to a fluid injection device for injecting a fluid behind a tympanic membrane of a patient, comprising a hollow needle having a needle tip with a fluid outlet at a distal end thereof, the needle tip being configured for piercing the tympanic membrane of the patient; a container for storing the fluid to be injected behind the tympanic membrane, the container being arranged in fluid communication with the hollow needle; a pump for pumping the fluid from the container into the hollow needle; and a grip, coupled to the hollow needle, preferably at or near a proximal end of the hollow needle, for holding the needle steadily and stably, wherein the grip comprises a touch button for activating the pump.

The fluid injection device according to the second aspect of the invention may, of course, also benefit from embodiments described in relation to the first aspect of the invention.

In an embodiment, the coupling between the hollow needle and the grip is of the rotational type, to allow a rotation of the grip with respect to the hollow needle. For example, said rotation may be over 120 or 180 degrees. Depending on the exact layout and configuration of the grip, allowing the needle to rotate may e.g. allow the same grip and, when present, the same touch button, to be used by left-handed as well as right-handed users, and/or may allow the same needle to be used for both the left ear as well as the right ear, and/or may allow the same needle to be used for ears with ear membranes, such as the tympanic membrane, positioned at different angles relative to the outer ear canal.

In an embodiment, the grip comprises a display arranged in communication with the container, the display being configured for displaying the amount of fluid injected with the fluid injection device. This provides an overview of the amount of fluid injected behind the tympanic membrane to the user of the fluid injection device, and provides full control to said user to start and stop the injection of fluid. The display may be arranged in communication with the container, but (when present) may alternatively and/or additionally also be arranged in communication with the pump.

As an alternative to a display, an audio signal may be generated based on the amount of fluid in the container and/or the working of the pump.

The use of a display in communication with the container is also advantageous in fluid injection devices that do not have elongated venting apertures in accordance with the first aspect of the invention.

Accordingly, a third aspect of the invention relates to a fluid injection device for injecting a fluid behind a tympanic membrane of a patient, comprising a hollow needle having a needle tip with a fluid outlet at a distal end thereof, the needle tip being configured for piercing the tympanic membrane of the patient; a container for storing the fluid to be injected behind the tympanic membrane, the container being arranged in fluid communication with the hollow needle and a grip, coupled to the hollow needle, preferably at or near a proximal end of the hollow needle, for holding the needle steadily and stably, wherein the grip comprises a display arranged in communication with the container, the display being configured for displaying the amount of fluid injected with the fluid injection device.

When present, the display may alternatively and/or additionally also be arranged in communication with a pump for pumping the fluid from the container into the hollow needle.

The fluid injection device according to the third aspect of the invention may, of course, also benefit from embodiments described in relation to the first aspect of the invention.

In an embodiment, the grip comprises two recesses for receiving, in use, a finger of a, and a line of sight from an eye of a user using the fluid injection device into the ear canal of the patient is defined between the two recesses. For example, the one recess may be a thumb receiving portion and the second recess may be an index finger receiving portion or a middle finger portion. For example, one may hold the grip with the thumb and the middle finger, and operate the touch button—when it is present—with the index finger. Alternative, one may hold the grip with the thumb and the index finger, and operate the touch button by a sliding motion of said index finger. When the line of sight is defined between the two recesses, this provides a user of the fluid injection device with a clear and unobstructed view into the outer ear canal on the eardrum of the patient, and thus allows to handle the fluid injection device with more precision, more stability and less vibrations. This reduces the chance of pain and damage to the ear.

In an embodiment, the fluid injection device further comprises an endoscope that is rigidly connected to at least a proximal portion of the needle, the endoscope being arranged in communication with a screen, so as to provide a visual representation of the needle tip and its surroundings on the screen via the endoscope. An endoscope, e.g. comprising a camera, preferably with a magnifying lens of e.g. between 4 and 15 diopters, and a light source, advantageously provides a clear view into the outer ear canal on the eardrum of the patient and allows the user of the fluid injection device to work with great precision. When the endoscope is rigidly connected to the proximal portion of the needle, the view provided by the endoscope ideally closely matches the physical position of the needle and/or needle tip, with a minimum amount of leeway. When the endoscope is arranged in communication with a screen, the user of the fluid injection device may not be limited to viewing directly in the outer ear canal of the patient, but may have a much clearer and larger view of this region on the screen. This additionally allows other persons to have the same view as the user of the fluid injection device, e.g. for training purposes or to assist in the making of decisions during the procedure.

The use of an endoscope in communication with a screen is also advantageous in fluid injection devices that do not have elongated venting apertures in accordance with the first aspect of the invention.

Accordingly, a fourth aspect of the invention relates to a fluid injection device for injecting a fluid behind a tympanic membrane of a patient, comprising a hollow needle having a needle tip with a fluid outlet at a distal end thereof, the needle tip being configured for piercing the tympanic membrane of the patient, a container for storing the fluid to be injected behind the tympanic membrane, the container being arranged in fluid communication with the hollow needle, a grip, coupled to the hollow needle, preferably at or near a proximal end of the hollow needle, for holding the needle steadily and stably, wherein an endoscope that is rigidly connected to a proximal portion of the needle, the endoscope being arranged in communication with a screen, so as to provide a visual representation of the needle tip and its surroundings on the screen via the endoscope.

The fluid injection device according to the fourth aspect of the invention may, of course, also benefit from embodiments described in relation to the first aspect of the invention.

The invention further relates to a hollow needle for use in a fluid injection device as described in the above, the hollow needle having a needle tip with a fluid outlet at a distal end thereof, the needle tip being configured for piercing a tympanic membrane of a patient, and comprising an elongated venting aperture in an outer wall thereof, the elongated venting aperture being positionable at an inner side of an ear membrane of the patient as well as at an outer side of said ear membrane when injecting fluid, so as to allow excess fluid on the inner side of said membrane to flow through the elongated venting aperture, to the outer side of said membrane.

The needle may e.g. be manufactured in the normal way (i.e. by deep drawing), wherein possibly a retractable protrusion on the die may create the elongated venting aperture during the deep drawing process. An alternative production method may be to etch material away from a “regular” needle, the etch defining the elongated venting aperture. A further alternative production method may be to engrave, grind, or mill a regular needle, the engraving, grinding or milling step fully removing a portion of the wall of the needle such that an aperture results.

The invention further relates to an injection assembly comprising a fluid injection device as described in the above and an injection fluid, wherein the injection fluid preferably has an intrinsic viscosity between 100 m³/kg and 500 m³/kg. The injection fluid will typically be stored in the container of the fluid injection device.

In an embodiment thereof, the fluid injection device of the injection assembly comprises a heating element for heating the injection fluid before the injection fluid is injected behind the tympanic membrane. In embodiments, said heating element heats the injection device to a temperature of between 25° C. (room temperature) and 50° C. , preferably within maximum plus or minus 7 degrees Celsius from body temperature, i.e. preferably between 30° C. and 42° C. However, in embodiments where the patient is e.g. under general anesthesia when the fluid is injected this may not be necessary. For example, some kinds of treatment fluids, e.g. when containing stem cells, cannot be heated to such temperature to not negatively affect the working of the treatment fluid. In such cases, bringing the patient under general anesthesia may be needed and the temperature of the injected fluid is not relevant. When a relatively cold or hot fluid is injected in the middle or inner ear of a patient this will cause nausea and vomiting. This reaction is prevented by heating the fluid to about body temperature. For example, the container may contain a heating element, in contact with the injection fluid and heating the injection fluid. In another example, the heating element is provided externally of the container, and heats the injection fluid by heating the container. In yet another embodiment, a tube between the needle and the container may contain a heating element that heats the injection fluid while it is transported from the container to the needle. This latter embodiment allows to store the fluid relatively cold, which may be desirable, while also preventing said nausea and vomiting reaction when the fluid is injected behind the tympanic membrane of the patient.

The invention further relates to a method for administering a fluid intratympanically, the fluid e.g. comprising a therapeutic agent, wherein use is made of the fluid injection device as described in the above for the administration of the fluid. The invention in particular further relates to an injection fluid, injected by the fluid injection device as described in the above, for use in preventing, alleviating or curing hearing loss, preferably sensorineural hearing loss, wherein the sensorineural hearing loss is induced by a platinum-based drug, particularly by cisplatin. Such a platinum-based drug is, for example, administered to a patient in the treatment of a cancer. The injection fluid preferably has an intrinsic viscosity between 100 m³/kg and 500 m³/kg. Said injection fluid preferably comprises a pharmaceutical component. In a preferred embodiment the pharmaceutical component is sodium thiosulphate (STS).

The skilled person is familiar with methods to determine the intrinsic viscosity of a liquid or a gel. A test method that can be used for determining the intrinsic viscosity of a composition, for example of the composition described herein, is a method for the Intrinsic Viscosity by Capillary flow according to Ph.Eur. 2.2.8./2.2.9 and further in the applicable sodium hyaluronate monograph (01/2017:1472). In said method the flow times of 4 dilutions of test material are determined in a suitable capillary in an appropriate suspended level viscometer. As provided herein the intrinsic viscosity is calculated by linear least squares regression analysis of flow times against the concentration of the samples using the Martin equation. The skilled person is well aware of other suitable test methods. The skilled person is familiar with similar methods of calculating intrinsic viscosity.

Further, as disclosed herein, viscosity is measured by equipment such as a Capillary Viscometer, for example a Capillary Viscometer Ubbelohde, or similar equipment known to a skilled person

These and other aspects of the present invention are described in more detail with reference to the attached figures. In these figures, the same reference numerals will be used for the same or like features. In the figures:

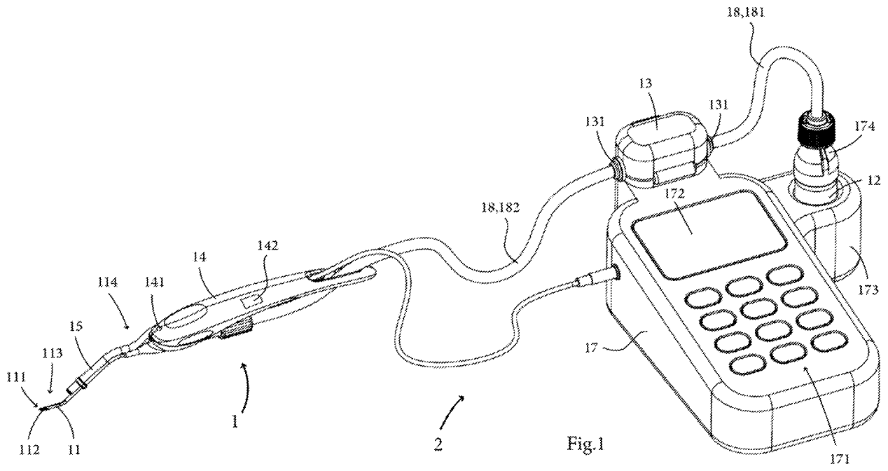

FIG. 1 schematically shows a schematic overview of a first embodiment of the fluid injection device according to the present invention;

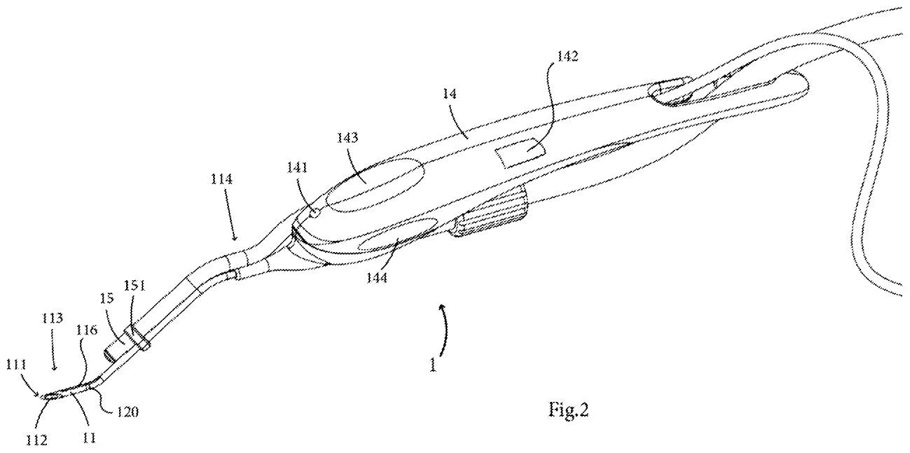

FIG. 2 schematically shows a detail of the fluid injection device of FIG. 1;

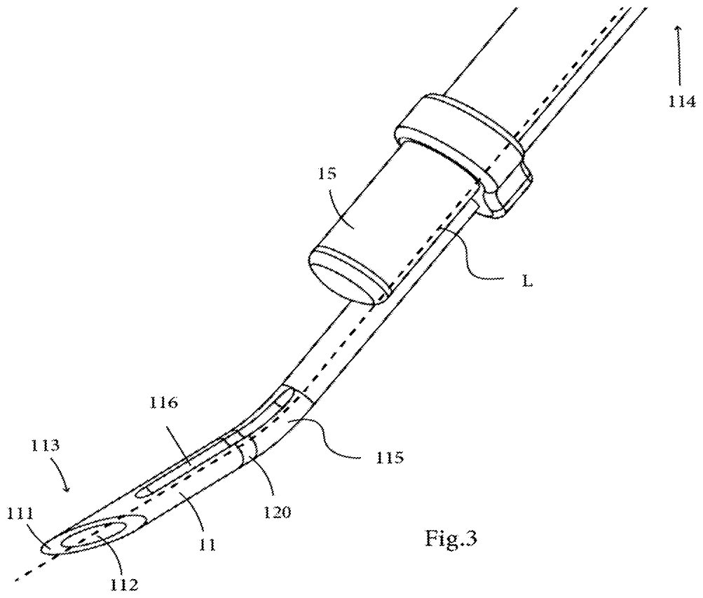

FIG. 3 schematically shows a detailed view of a needle of the fluid injection device of FIG. 1;

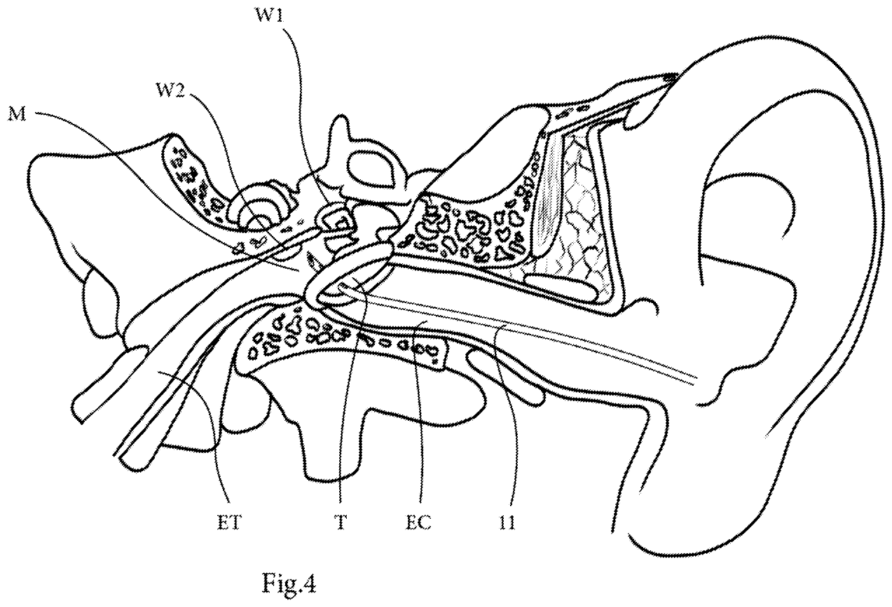

FIG. 4 schematically shows a cross-sectional view of an ear of a human, with a needle of a fluid injection device according to the invention inserted in said ear;

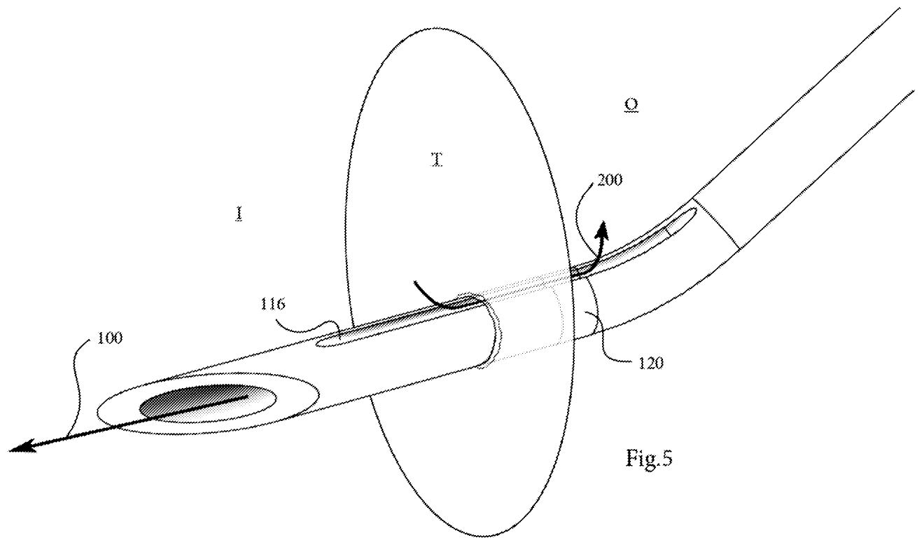

FIG. 5 schematically shows a detailed view of a needle tip of a needle of a fluid injection device according to the invention, penetrating a tympanic membrane;

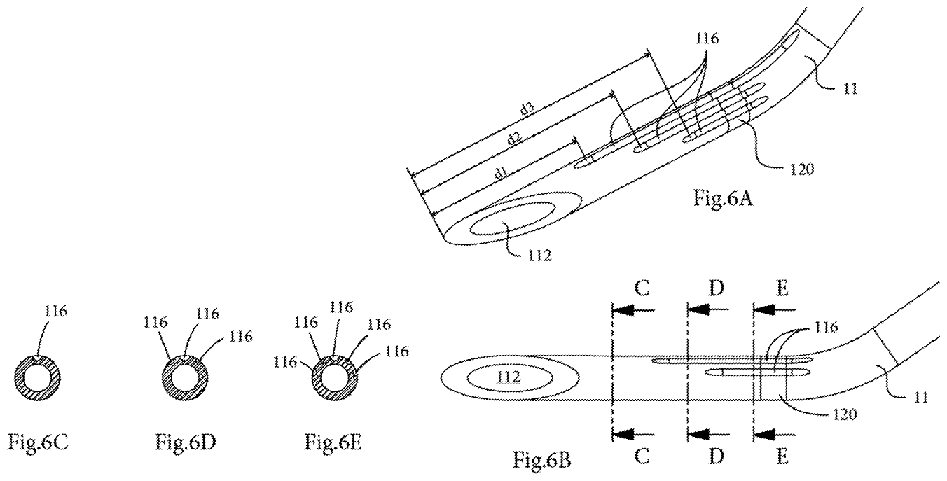

FIGS. 6A-6E schematically show different views of a second embodiment of a needle of a fluid injection device according to the invention;

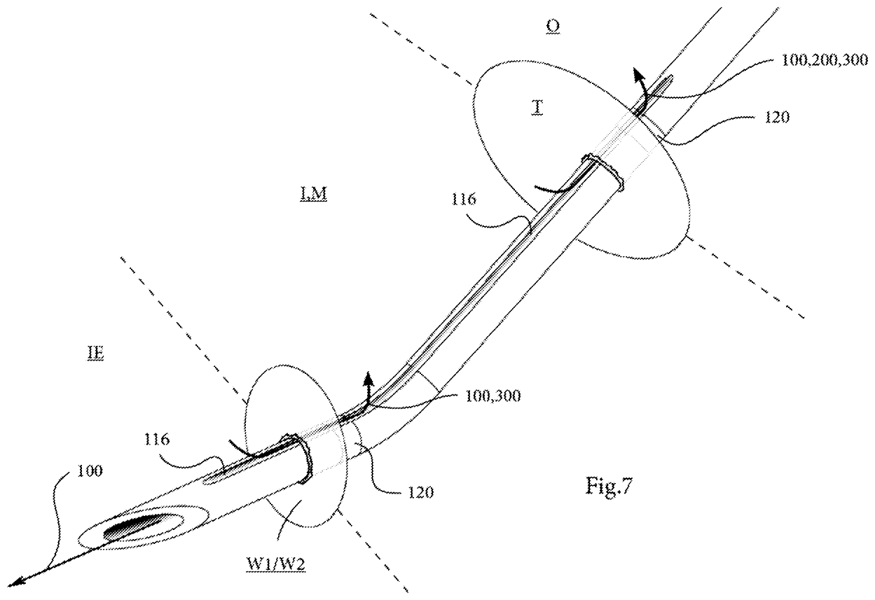

FIG. 7 schematically shows a detailed view of a needle tip of a needle of a third embodiment of a fluid injection device according to the invention, penetrating both the tympanic membrane and the oval or round window; and

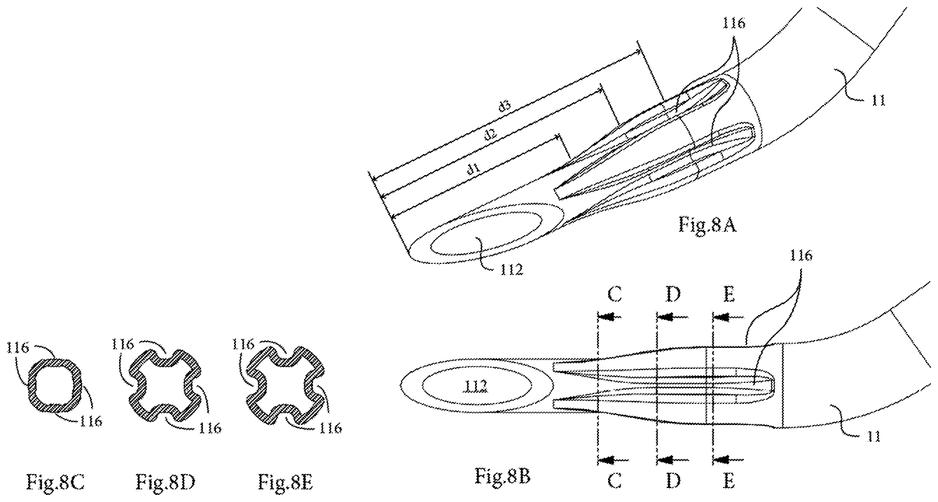

FIGS. 8A-8E schematically show different views of a fourth embodiment of a needle of a fluid injection device according to the invention.

With respect to FIG. 1, a fluid injection device 1 and a fluid injection assembly 2 are shown. The fluid injection device 1 is configured for injecting a fluid behind a tympanic membrane of a patient, as will be explained in more detail with reference to FIGS. 4 and 5. The embodiment of the fluid injection device 1 shown in FIG. 1 comprises a base unit 17, that may e.g. comprise buttons 171, a screen 172 and software for operating the fluid injection device 1. Via the buttons 171 input may be provided to the software running the fluid injection device 1 and via screen 172 information about the working of the fluid injection device 1 may be displayed to a user thereof. The base unit 17 further comprises a holder 173 in which a container 12 may be placed. The fluid which is to be injected in an ear of a patient with the fluid injection device 1 is stored in said container 12. The container 12 is releasably coupled to the base unit 17 via cap 174. While the cap 174 is part of the base unit 17, the container 12 is replaceable. However, the cap 174 may also be replaceable, e.g. along with the tubing, to prevent any cross-contamination when the base unit 17 is re-used.

Via cap 174, tubing 18 and pump 13 the fluid in the container 12 is arranged in fluid communication with a needle 11. When the pump 13 is operated, it sucks fluid out of the container 12, through a first portion 181 of the tubing 18, through the pump 13 itself and displaces the fluid into the needle 11 via a second portion 182 of the tubing 18. The second tubing portion 182, arranged between the pump 13 and the needle 11, is rigidly clamped to the pump via one or two clamping members 131. This prevents any vibrations from operation of the pump 13 to be transferred to the needle 11, and thus results in a safer working of the fluid injection device 1. The clamping member(s) 131 preferably also prevent movement of the tube section inside the peristaltic pump, because movement of this tubing could affect the dose accuracy.

The tubing 18 shown in relation to the embodiment of FIG. 1 is relatively long, which results in a relatively large amount of injection fluid remaining present in the tubing 18 and not injected in the ear of the patient. For relatively inexpensive injection fluids this is fine. However, it may also be desired to inject relatively expensive, rare or hard to obtain fluid in the ear of a patient. In that case, a large amount of injection fluid remaining in the tube 18 and not injected in the ear is undesirable. In such an application, the skilled person will have no problem to change the design of the shown fluid injection device and reduce the distance between the container and the needle. For example, the container 12 storing the injection fluid may be arranged in between the pump 13 and the needle 11, close to the needle 11.

The fluid stored in the container 12 preferably comprises a therapeutic agent and preferably has a relatively high viscosity to allow the fluid to be optimally received and contained in the middle or inner ear of the patient. For example, the intrinsic viscosity of the fluid may be between 100 m³/kg-500 m³/kg.

Moving to FIG. 2, the grip 14 and the needle 11 of the fluid injection device 1 are shown in more detail. The grip 14 as shown here has an index finger receiving portion 143 and a thumb receiving portion 144, and is arranged at a proximal end 114 of the needle 11. Above the index finger receiving portion 143 a touch button 141 is arranged which may be operated with an index finger of a user of the fluid injection device. For example, the touch button 141 may be arranged in communication with the pump, and may activate and/or stop the working of said pump. The index finger receiving portion 143 and the thumb receiving portion 144 are designed and placed to allow a user of the fluid injection device to hold the grip stably and steadily. When using the fluid injection device 1, a user has a clear view into the ear of the patient when a line of sight between the eye of the user and the ear canal of the patient is defined between said index finger receiving portion 143 and said thumb receiving portion 144.

Further shown in FIG. 2 is a display 142. The display 142 is arranged in communication with the pump and/or the container and displays 142 information regarding the amount of fluid injected with the fluid injection device. This directly indicates to a user of the fluid injection device when to stop the injection process without needing to rely on information of a second person and/or other possibly erroneous information sources.

FIG. 2 further shows an endoscope 15, rigidly connected to the needle 11 via clamp 151. The endoscope 15 provides a view inside the ear canal, but outside of the ear membrane, of the patient to a user of the fluid injection device. The endoscope 15 is preferably arranged in communication with a (non-shown) screen, e.g. via a wireless communication source, so as to provide a visual representation of the surroundings of a needle tip 111 and the needle tip 111 itself.

With reference to FIG. 3, one embodiment of a needle 11 of the fluid injection device is shown. As shown here, the needle 11 comprises a needle tip 111, slanted to allow a piercing of at least the tympanic membrane of a patient, a fluid outlet 112, an outer wall 115 and an elongated venting aperture 116. The needle 11 here comprises only a single wall, the outer wall 115 and is single-walled. As shown, the needle 11 is bent, such that a distal end 113 of the needle 11 is inclined with respect to a proximal end 114 of the needle 11. The bending of the distal end 113 may make it easier to a user of the fluid injection device to penetrate an ear membrane of a patient at a right angle—a right angle being least likely to rupture said membrane. A longitudinal direction L of the needle 11 follows the bend.

The distal portion 113 of the needle 11 comprises a positioning indicator 120 that indicates a pre-defined insertion depth of the needle 11. Depending on instruction provided along with the fluid injection device, the needle 11 is e.g. to be inserted through the ear membrane of the user until the positioning indicator 120 disappears from sight, or the positioning indicator 120 marks the position until which the needle tip 111 may be inserted through the ear membrane. Seen in a longitudinal direction L of the needle 111, in which direction the elongated venting aperture 116 is arranged, the positioning indicator 120 overlaps with the elongated venting aperture 116.

Compared to the needle 11 of FIG. 3, the needle of FIGS. 6A-6E shows an alternative embodiment thereof. The needle 11 shown in FIGS. 6A-6E comprises a total of five elongated venting apertures 116, arranged at three different distances dl, d2, d3 of the needle tip 111. Depending on the insertion depth of the needle 11, more or fewer venting apertures extend through the ear membrane of the patient, and more or less venting functionality is achieved. When the ear membrane is positioned at the cross-sectional plane of FIG. 6C, only a relatively small amount of venting is achieved, as only one venting aperture 116 is functional.

When the ear membrane is positioned at the cross-sectional plane of FIG. 6D, some more venting is achieved, as now three venting apertures 116 are functional. If the ear membrane would be positioned at the cross-sectional plane of FIG. 6E, the maximum amount of venting is achieved. Therefore, advantageously, when the needle 11 comprises several elongated venting apertures 116, arranged at different distances from the needle tip 111, the user of the fluid injection device may select the desired amount of venting and/or change the amount of venting while injecting fluid.

Compared to the needle 11 of FIGS. 6A-6E, the needle 11 shown in FIGS. 8A-8E shows an alternative solution to vary the amount of venting based on the insertion depth of the needle 11 in an ear membrane. As shown in FIGS. 8A-8E venting apertures 116 are gradually introduced in the outer wall of the needle 11. This may be effected by a double-walled solution, wherein the outer wall provides the venting effect and the inner wall provides an injection channel and outlet 112. However, the needle 11 as shown in FIGS. 8A-8E can also be made using only a single wall. As shown by the different cross-sectional views in FIGS. 8C-8E, the further the needle 11 is inserted in an ear membrane, the more venting is allowed. A particular advantage of the needle 11 shown in FIGS. 8A-8E is that the outer diameter of the needle 11 gradually increases in a direction from the needle tip towards the end of the venting apertures 116. This may further help in preventing pain and/or barotrauma.

Insertion of the needle 11 of the fluid injection device and penetration of the needle 11 through an ear membrane of a patient is illustrated with reference to FIGS. 4 and 5, to be discussed together. FIG. 4 schematically shows a cross-sectional view of an ear of a human. The ear comprises an external ear channel EC, a tympanic membrane T, an Eustachian tube ET, an oval window W1, and a round window W2. The middle ear M is generally defined between the tympanic membrane T and the round W2 and oval window W1. The inner ear is generally defined beyond the round W1 and oval window W2. In FIG. 4, the needle 11 is shown while it is partially penetrated through the tympanic membrane T, extending into the middle ear M.

In FIG. 5 a more detailed view of the needle tip, penetrating the tympanic membrane T, is provided. As shown, an elongated venting membrane 116 is arranged partially inside the tympanic membrane T and partially outside the tympanic membrane T. As schematically shown, fluid 100 is injected at an inside I of the tympanic membrane T, i.e. behind the tympanic membrane T, with the needle. As a result of this injection, the pressure at the inside I of the tympanic membrane T, in the middle ear, rises and the fluid residing there before injection of fluid, e.g. air, wants to move out of the middle ear. This is made possible by the elongated venting aperture 116, which provides a venting channel from the inside I of the tympanic membrane T to the outside O of the tympanic membrane T. The fluid 200 thus moves through this venting channel from inside the ear membrane T to outside of the ear membrane T.

Comparing FIG. 7 to FIG. 5, in FIG. 7 a needle 11 is shown which penetrates both the tympanic membrane T and the oval window W1 or the round window W2, such that an outlet of the needle 11 reaches the inner ear IE. As shown here, an elongated venting membrane 116 is arranged partially inside the tympanic membrane T, at the inside I of the ear, more particularly at the middle ear M, and partially outside the tympanic membrane T at the outside O of the ear. The elongated venting membrane 116 is also arranged partially inside the round window W2 or the oval window W1, in the inner ear IE, and partially outside the round window W2 or the oval window W1, in the middle ear M. As shown here the elongated venting aperture 116 extends all the way from the inner ear IE to the outside O of the ear. In alternative embodiments, there are two elongated venting apertures, one for penetrating the oval or round window and one for penetrating the eardrum T. As schematically shown, fluid 100 is injected at in the inner ear IE with the needle Compared to the outside O of the ear, the inner ear IE is arranged at the inner side I of the tympanic membrane T. As a result of this injection, the pressure in the inner ear IE, rises and the fluid 300 residing there before injection of fluid 100, wants to move out of the inner ear IE. This is made possible by the elongated venting aperture 116, which provides a venting channel from the inner ear IE to the middle ear M (i.e. from the inner side with respect to the oval or round window to the outer side with respect to the oval or round window). The fluid 300 thus moves through this venting channel from inside the ear membrane W1, W2 to outside of the ear membrane W1, W2.

As a result of the fluid 300 moving in the middle ear M, the pressure at the inside I of the tympanic membrane T, may rise and the fluid residing there may want to move out of the middle ear. This is also made possible by the elongated venting aperture 116, which additionally provides a venting channel from the inside I of the tympanic membrane T to the outside O of the tympanic membrane T. The fluid 200 thus moves through this venting channel from inside the ear membrane T to outside of the ear membrane T.

As an alternative to the solution presented in FIG. 7, of course also the conventional technique to inject fluid in the inner ear may be used without departing from the inventive concept as described herein. According to this conventional technique, the tympanic membrane is not penetrated but circumcised and folded aside to expose the middle ear. With the middle ear exposed in this way, the surgeon can access the round and/or oval window to the inner ear and pierce it with the needle described herein. This procedure is typically performed under a general anesthesia.

### LIST OF REFERENCE NUMERALS

1 fluid injection device
11 hollow needle

111 needle tip

112 fluid outlet

113 distal end

114 proximal end

115 outer wall

116 venting aperture

117 inner wall

118 injection channel

119 venting channel

120 positioning indicator
12 container
13 pump

131 clamping member
14 grip

141 touch button

142 display
15 endoscope

151 clamp
17 base unit

171 buttons

172 screen

173 holder

174 cap
18 tubing

181 first tubing portion

182 second tubing portion

2 fluid injection assembly
100 injection fluid
200 middle ear fluid
300 inner ear fluid
d1 distance between fluid outlet and first venting aperture
d2 distance between fluid outlet and second venting aperture
d3 distance between fluid outlet and third venting aperture
EC ear channel
ET Eustachian tube
I inside of ear
IE inner ear
O outside of ear
L longitudinal direction needle
M middle ear
P patient
T tympanic membrane
W1 oval window
W2 round window

1. A fluid injection device for injecting a fluid behind a tympanic membrane of a patient, comprising:
a hollow needle having a needle tip with a fluid outlet at a distal end thereof, the needle tip being configured for piercing the tympanic membrane of the patient;
a container for storing the fluid to be injected behind the tympanic membrane, the container being arranged in fluid communication with the hollow needle;
wherein the hollow needle comprises an elongated venting aperture in an outer wall thereof, the elongated venting aperture being positionable at an inner side of an ear membrane of the patient as well as at an outer side of said ear membrane when injecting fluid, so as to allow excess fluid on the inner side of said membrane to flow through the elongated venting aperture, to the outer side of said membrane.

2. The fluid injection device according to claim 1, wherein the hollow needle comprises at least two elongated venting apertures, and wherein a distance between a first elongated venting aperture and the fluid outlet differs from a distance between a second elongated venting aperture and the fluid outlet.

3. The fluid injection device according to claim 1, wherein the venting aperture is arranged in the longitudinal direction of the needle.

4. The fluid injection device according to claim 1, wherein the hollow needle is a single-walled needle.

5. The fluid injection device according to claim 1, wherein the hollow needle is a double-walled needle having an inner wall and an outer wall, a fluid injection channel being defined by the inner wall and a venting channel being defined between the outer wall and the inner wall.

6. The fluid injection device according to claim 1, wherein the needle, at or near an area where the venting aperture is arranged, comprises a positioning indicator for indicating a pre-defined insertion depth of the needle.

7. The fluid injection device according to claim 1, further comprising a grip releasably coupled to the hollow needle at or near a proximal end of the hollow needle, for holding the needle.

8. The fluid injection device according to claim 7, wherein the fluid injection device further comprises a pump for pumping the fluid from the container into the hollow needle, wherein the grip comprises a touch button for activating the pump.

9. The fluid injection device according to claim 8, wherein the coupling between the hollow needle and the grip is of the rotational type, to allow a rotation of the grip with respect to the hollow needle.

10. The fluid injection device according to claim 7, wherein the grip includes a display arranged in communication with the container, the display being configured for displaying the amount of fluid injected with the fluid injection device.

11. The fluid injection device according to claim 7, wherein the grip comprises two recesses for receiving, in use, a finger of a user, and a line of sight from an eye of a user using the fluid injection device into the ear canal of the patient is defined between the two recesses.

12. The fluid injection device according to claim 1, further comprising an endoscope that is rigidly connected to at least a proximal portion of the needle, the endoscope being arranged in communication with a screen, so as to provide a visual representation of the needle tip and its surroundings on the screen via the endoscope.

13. A hollow needle for use in a fluid injection device, the hollow needle having a needle tip with a fluid outlet at a distal end thereof, the needle tip being configured for piercing a tympanic membrane of a patient, and comprising an elongated venting aperture in an outer wall thereof, the elongated venting aperture being positionable at an inner side of an ear membrane of the patient as well as at an outer side of said ear membrane when injecting fluid, so as to allow excess fluid on the inner side of said membrane to flow through the elongated venting aperture, to the outer side of said membrane.

14. An injection assembly comprising a fluid injection device according to claim 1 and an injection fluid, wherein the injection fluid preferably has an intrinsic viscosity between 100 m³/kg and 500 m³/kg.

15. The injection assembly according to claim 14, wherein the fluid injection device comprises a heating element for heating the injection fluid before the injection fluid is injected behind the tympanic membrane.

16. The fluid injection device according to claim 1, comprising an injection fluid for use in preventing, alleviating or curing hearing loss.

17. The fluid injection device according to claim 1, wherein the injection fluid comprises a pharmaceutical component.

18. The fluid injection device according to claim 1, wherein the pharmaceutical component is sodium thiosulphate.

19. The fluid injection device according to claim 1, wherein the injection fluid has an intrinsic velocity between 100 m³/kg and 500 m³/kg.

20. The fluid injection device according to claim 16, wherein the hearing loss is sensorineural hearing loss or is induced by a platinum-based drug.

21. (canceled)
